(19) United States
(12) Patent Application Publication
(10) Pub. No.: US 2024/0050358 A1
(43) Pub. Date: Feb. 15, 2024

(54) PERSONAL CARE COMPOSITION

(71) Applicant: Conopco, Inc., d/b/a UNILEVER, Englewood Cliffs, NJ (US)

(72) Inventors: Xuelan GU, Shanghai (JP); Dandan HUANG, Shanghai (CN); Hong ZHANG, Shanghai (CN)

(73) Assignee: Conopco, Inc., d/b/a UNILEVER, Englewood Cliffs, NJ (US)

(21) Appl. No.: 18/265,324

(22) PCT Filed: Dec. 6, 2021
(86) PCT No.: PCT/EP2021/084300

§ 371 Date: Jun. 5, 2023

(30) Foreign Application Priority Data
Dec. 25, 2020 (WO) PCT/CN2020/139451
Jan. 28, 2021 (EP) 21153894.7

(51) Int. Cl. A61K 8/9789; A61K 8/365; A61K 8/49; A61K 8/34; A61Q 19/08

(52) U.S. Cl. CPC A61K 8/9789; A61K 8/365; A61K 8/4926; A61K 8/347; A61Q 19/08

(57) ABSTRACT

Disclosed is a personal care composition providing anti-inflammation benefits. The composition comprises a Moringa extract and a hydroxy fatty acid, wherein the weight ratio of the Moringa extract to the hydroxy fatty acid is from 1:1 to 75:1.

### FIELD OF THE INVENTION

The present invention relates to a personal care composition. In particular, the present invention relates to a personal care composition for providing improved anti-inflammatory efficacy.

### BACKGROUND OF THE INVENTION

Inflammation is a complex biological host response to harmful stimuli such as pathogens, irritants or injury. Inflammation is also a protective mechanism by which the host removes the stimuli and begins the healing process.

There are generally two types of inflammation, namely acute inflammation and chronic inflammation. Acute inflammation is a rapid process characterized by fluid exudation and emigration of leukocytes, primarily neutrophils, whereas chronic inflammation extends over a longer time and is associated with lymphocyte and macrophage infiltration, blood vessel proliferation, and fibrosis.

Inflammation is closely related to various skin problems, for example, acne, post-inflammatory hyperpigmentation (PIH) and ageing. Acne, also known as Acne vulgaris, is a common inflammatory skin problem which affects the pilosebaceous units of the skin. It can leave the subjects with severe skin scarring and also have severe psychological effects.

PIH is a hyperpigmentation of the skin occurring during or after an inflammatory process. PIH can be observed in any skin types and at all ages. In most case, PIH exerts a strong impact on the quality of life of affected individuals. External insults such as ultraviolet light exposure could also cause inflammation, which may enhance skin aging even further leading to signs of photoaging.

A few approaches have been taken to cope with inflammation-related problems, including topical and systemic treatments using antibiotics and steroids. However, those treatments are either irritating, causing resistance or having some other side effects.

Therefore, people are continuously developing new solutions for anti-inflammatory benefits.

The present inventors have surprisingly found that a personal care composition comprising Moringa extract and hydroxy fatty acid provides synergistic anti-inflammatory benefit.

### SUMMARY OF THE INVENTION

In a first aspect, the present invention provides a personal care composition comprising:
(a) a Moringa extract, and
(b) a hydroxy fatty acid having 8 to 24 carbon atoms,
wherein the weight ratio of the Moringa extract to the hydroxy fatty acid is 1:1 to 75:1.

In a second aspect, the present invention provides a method for providing at least one benefits selected from anti-inflammation, anti-aging, and anti-acne comprising the step of applying the composition of the present invention to the skin of an individual.

In a third aspect, the present invention provides use of the composition of the present invention for providing at least one benefits selected from anti-inflammation, anti-aging, and anti-acne.

All other aspects of the present invention will more readily become apparent upon considering the detailed description and examples which follow.

### DETAILED DESCRIPTION OF THE INVENTION

Except in the examples, or where otherwise explicitly indicated, all numbers in this description indicating amounts of material or conditions of reaction, physical properties of materials and/or use may optionally be understood as modified by the word “about”.

All amounts are by weight of the composition, unless otherwise specified.

It should be noted that in specifying any range of values, any particular upper value can be associated with any particular lower value.

For the avoidance of doubt, the word “comprising” is intended to mean “including” but not necessarily “consisting of” or “composed of”. In other words, the listed steps or options need not be exhaustive.

The disclosure of the invention as found herein is to be considered to cover all embodiments as found in the claims as being multiply dependent upon each other irrespective of the fact that claims may be found without multiple dependency or redundancy.

The composition of the present invention comprises a Moringa extract. Typically, the Moringa extract is extracted from seeds, fruits, leaves and/or bark of Moringa, preferably from seeds or fruits of Moringa, and more preferably from seeds of Moringa selected from Moringa oleifera Lam., Moringa pterygosperma, Moringa peregrina, Moringa concanensis and Moringa drouhardii, even more preferably from seeds of Moringa selected from Moringa oleifera Lam. and Moringa pterygosperma. Preferably, the Moringa extract is extract by aqueous solution.

Preferably, the Moringa extract is employed in the composition in an amount of 0.00001 to 1% by weight of the composition, more preferably in an amount of 0.0001 to 0.5%, even more preferably from 0.0005 to 0.2% and most preferably in an amount of 0.001 to 0.05% by weight of the composition.

The composition of the present invention further comprises a hydroxy fatty acid. Fatty acids are carboxylic acids with a long aliphatic chain which usually has 6 to 30 carbon atoms. Hydroxy fatty acids are derivatives of fatty acids containing a hydroxyl group at one or more positions. Preferably the fatty acids are saturated or unsaturated and branched or unbranched. Preferably, the hydroxy fatty acids used in the present invention have 8 to 24 carbon atoms, preferably 10 to 22 carbon atoms, more preferably 16 to 20 carbon atoms, most preferably 18 to 20 carbon atoms.

Preferably, the hydroxy fatty acid is selected from 2-hydroxymyristic acid, 3-hydroxymyristic acid, 2-hydroxypalmitic acid, 3-hydroxypalmitic acid, 12-hydroxystearic acid, 17-hydroxystearic acid, and mixture thereof.

Preferably, the hydroxy fatty acid is hydroxystearic acid. The most preferred hydroxy fatty acid is 12-hydroxystearic acid (12HSA).

Preferably, the amount of hydroxy fatty acid is in the range of 0.000001 to 5%, more preferably 0.00001 to 1%, even more preferably 0.00005 to 0.2%, and still even more preferably 0.0001 to 0.01% by weight of the composition.

Preferably, the weight ratio of the Moringa extract to the hydroxy fatty acid is from 1:1 to 75:1, more preferably from 2:1 to 60:1, and more preferably from 15:1 to 60:1.

The composition preferably comprises one or more organic sunscreens. A wide variety of organic sunscreen is suitable for use in combination with the essential ingredients of this invention. Suitable organic sunscreen includes, 2-hydroxy-4-methoxybenzophenone, octyldimethyl p-aminobenzoic acid, digalloyltrioleate, 2,2-dihydroxy-4-methoxybenzophenone, ethyl-4-(bis(hydroxypropyl)) aminobenzoate, 2-ethylhexyl-2-cyano-3,3-diphenylacrylate, 2-ethylhexylsalicylate, glyceryl p-aminobenzoate, 3,3,5-trimethylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, 2-ethylhexyl-p-methoxycinnamate, butylmethoxydibenzoylmethane, 2-hydroxy-4-methoxybenzophenone, octyldimethyl-p-aminobenzoic acid and mixtures thereof. The most suitable organic sunscreens are 2-ethylhexyl-p-methoxycinnamate, butylmethoxydibenzoylmethane or a mixture thereof.

A safe and effective amount of organic sunscreen may be used in the compositions useful in the subject invention. The composition preferably comprises from 0.1 to 10%, more preferably from 0.1 to 5%, of organic sunscreen by weight of the composition.

Compositions of the present invention may comprise a cosmetically acceptable carrier. Oily carriers in the presence of water and an emulsifier will form emulsion systems as carriers. These systems may either be water-in-oil or oil-in-water emulsions. Besides water, suitable carrier classes include silicones, hydrocarbons, polyhydric alcohols, fatty alcohols, triglycerides or a mixture thereof.

Silicones when present may range from 5 to 60%, more preferably from 5 to 40%, by weight of the composition. These silicones may be organic, silicone-containing or fluorine-containing, volatile or non-volatile, polar or non-polar.

Particularly preferred volatile silicone oils are cyclic volatile silicones wherein the repeating unit ranges from 3 to 5; and linear silicones wherein the repeating unit ranges from 1 to 7. Highly preferred examples of volatile silicone oils include cyclomethicones of varying viscosities, e.g., Dow Corning 200, Dow Corning 244, Dow Corning 245, Dow Corning 344 and Dow Corning 345 (commercially available from Dow Corning Corp.); SF-1204 and SF-1202 Silicone Fluids, GE 7207 and 7158 (commercially available from G.E. Silicones) and SWS-03314 (commercially available from SWS Silicones Corp.

Hydrocarbons may include mineral oil, petrolatum and polyalpha-olefins. Examples of preferred volatile hydrocarbons include polydecanes such as isododecane and isodecane (e.g. Permethyl-99A which is available from Presperse Inc.) and the C7-C8 through C12-C15 isoparaffins (such as the Isopar Series available from Exxon Chemicals).

Polyhydric alcohols may include propylyene glycol, dipropylene glycol, polypropylene glycol, polyethylene glycol, sorbitol, hydroxypropyl sorbitol, hexylene glycol, 1,3-butylene glycol, isoprene glycol, ethoxylated glycerol, propoxylated glycerol and mixtures thereof. Most preferred is glycerol known also as glycerin. The amount of polyhydric alcohol may range anywhere from 0.1 to 10%, preferably between 0.4 and 5% by weight of the composition.

Fatty alcohols may also be present. The term “fatty alcohols” refers to alcohols having carbon chain with lengths ranging from 10 to 30 carbon atoms. Illustrative of this category are lauryl alcohol, cetyl alcohol, stearyl alcohol, isostearyl alcohol and combinations thereof. Cetyl alcohol is more preferable.

Illustrative triglycerides but not limiting are sunflower seed oil, cotton oil, canola oil, soybean oil, castor oil, borage oil, olive oil, shea butter, jojoba oil and mixtures thereof. Mono- and di-glycerides may also be useful. Particularly preferable are glyceryl monostearate and glyceryl distearate.

The composition may comprise water in amount of 10 to 90% by weight of the composition, preferably from 35 to 85%, more preferably between 40 and 70% by weight of the composition.

The composition may comprise optional ingredients including skin glowing agent, preservatives, antioxidants, colorants, fragrance, or a combination thereof.

The composition of the invention may preferably further comprise a skin glowing agent. Vitamin B3 compounds (including derivatives of vitamin B3) e.g. niacin, nicotinic acid or niacinamide are the preferred skin glowing agent as per the invention, most preferred being niacinamide. Vitamin B3 compounds, when used, are preferably present in an amount in the range of 0.1 to 10%, more preferably 0.2 to 5% by weight of the composition.

Preferably, the personal care composition is a skin care composition. The skin care composition refers to a composition suitable for topical application to human skin. The term “skin” as used herein includes the skin on the face (except eye lids and lips), neck, chest, abdomen, back, arms, under arms, hands, and legs. Preferably “skin” means includes the skin on the face (except eye lids and lips) and under arms, more preferably skin means skin on the face other than lips and eyelids.

“Viscosity” as used herein means dynamic viscosity at 25° C. and is reported as mPa·s. A suitable method of measuring the viscosity is to use a stress-controlled MCR 501 rheometer (Anton Paar, Physica MCR501, Austria), fitted with a sandblast parallel geometry (PP25s), at shear rate 3.98 1/s. The viscosity of the composition suitably ranges from 1,000 to 20,000 mPa·s, preferably from 2,000 to 15,000 mPa·s, even more preferably from 2,500 to 12,000 mPa·s.

Preferably, the method is non non-therapeutic. Preferably the use is non-therapeutic. The term non-therapeutic means for cosmetic purposes and not curative or therapeutic purposes.

The present invention also provides a personal care composition a Moringa extract and a hydroxy fatty acid having 8 to 24 carbon atoms, wherein the weight ratio of the Moringa extract to the hydroxy fatty acid is 1:1 to 75:1 for use in providing at least one benefits selected from anti-inflammation, anti-aging, and anti-acne.

The present invention also provides use of a combination of a Moringa extract and a hydroxy fatty acid having 8 to 24 carbon atoms, wherein the weight ratio of the Moringa extract to the hydroxy fatty acid is 1:1 to 75:1 for usein the manufacture of a medicament for providing at least one benefit selected from anti-inflammation, anti-aging, and anti-acne.

The following examples are provided to facilitate an understanding of the invention. The examples are not intended to limit the scope of the claims.

### EXAMPLES

Moringa seed extract used in the examples was procured from WuXi AppTec Co., Ltd. (Shanghai, China).

### Human Immune Cell Line THP-1 In Vitro Assay

The following procedure was used to determine the anti-inflammation efficacy:

THP1-XBlue™ (Cat No.: thpx-sp, InvivoGen, San Diego, CA) cells were cultured as suspense in RPMI 1640 medium supplemented with 10% fetal bovine serum (FBS), penicillin (10 U/mL)-streptomycin (10 μg/mL) (Gibco, Invitrogen, Carlsbad, CA). Cells were differentiated in 24-well plates at the density of 5×10⁵ cells/well with 100 nM phorbol-12-myristate-13-acetate (PMA) for 72 hours. Cells were then co-treated with pure E. coli lipopolysaccharides (LPS) and various compositions in Table 1. After 24 hours, the supernatants were collected and measured for interleukin (IL)-6 as a pro-inflammatory biomarker using enzyme-linked immunosorbent assay (ELISA). Expression of IL-6 was calculated as the percentage to LPS-treated cells which was designated as 100%. P value was analysed by Student's t-test comparing combination groups and individual compound groups (expression of IL-6, three repeats for each group). P value <0.05 indicates synergistic effect.

The results in terms of expression of IL-6 in percentage are given in Table 1:

TABLE 1

| | | Expression | Std. | P |
|---|---|---|---|---|
| Examples | Composition | of IL-6 (%) | dev | value |
| A | LPS | 100 | 0 | — |
| B | 12HSA (3 ppm) | 91.9 | 5.8 | — |
| C | Moringa extract (100 ppm) | 15.2 | 2.8 | — |
| 1 | Moringa extract (100 ppm) + 12HSA (3 ppm) | 8.6 | 1.2 | 0.04* |
| D | Moringa extract (10 ppm) | 89.4 | 1.3 | — |
| 2 | Moringa extract (10 ppm) + 12HSA (3 ppm) | 83.3 | 0.7 | 0.01** |
| E | Moringa extract (300 ppm) | 1.6 | 0.3 | — |
| F | Moringa extract (300 ppm) + 12HSA (3 ppm) | 2.1 | 0.4 | 0.4 |
| G | Moringa extract (1 ppm) | 87.9 | 2.1 | — |
| H | Moringa extract (1 ppm) + 12HSA (3 ppm) | 83.1 | 2.5 | 0.2 |

*Example 1 vs. Example C
**Example 2 vs. Example D

From the above table it is evident that the examples within the scope of the present invention (Example 1 and 2) provide synergistic inhibition of IL-6 in THP-1 cells when compared with the examples that are outside the scope of the present invention (Example F and H).

Therefore, from the above description it is evident that the present invention provides a personal care composition that inhibits IL-6 and thereby reduce inflammation on the skin of individuals.

1. A personal care composition comprising:
(a) 0.00001 to 1% of Moringa extract by weight of the composition; and
(b) 0.000001 to 5% of hydroxy fatty acid having 8 to 24 carbon atoms by weight of the composition; and
wherein the Moringa extract to the hydroxy fatty acid is present in a weight ratio ranging from 1:1 to 75:1,
and further wherein the Moringa extract is extracted from Moringa selected from Moringa oleifera Lam., Moringa ptetygosperma, Moringa peregrina, Moringa concanensis and Moringa drouhardii.

2. The personal care composition as claimed in claim 1, wherein the Moringa extract to the hydroxy fatty acid is present in a weight ratio ranging from 2:1 to 60:1.

3. The personal care composition as claimed in claim 1, wherein the Moringa extract is extracted from Moringa selected from Moringa oleifera Lam. and Moringa pterygosperma.

4. The personal care composition as claimed in claim 1, wherein the Moringa extract is extracted from seeds, fruits, leaves and/or bark of Moringa.

5. The personal care composition as claimed in claim 1, wherein the amount of the hydroxy fatty acid is in the range of 0.00005 to 0.2 by weight of the composition.

6. The personal care composition as claimed in claim 1, wherein the amount of the Moringa extract is in the range of 0.0001 to 0.5% by weight of the composition.

7. The personal care composition as claimed in claim 1, wherein the hydroxy fatty acid is hydroxystearic acid.

8. The personal care composition as claimed in claim 1, wherein the composition further comprises an additional skin benefit agent selected from niacinamide, picolinamide, isonicotinamide, hexyl resorcinol, or a mixture thereof.

9. The personal care composition as claimed in claim 1, wherein the composition comprising water.

10. The personal care composition as claimed in claim 1, wherein the viscosity of the composition is in the range of 1,000 to 20,000 mPa·s.

11. A method for providing at least one benefit selected from anti-inflammation, anti-aging, and anti-acne comprising the step of applying the personal care composition as claimed in claim 1, to the skin of an individual.

12. (canceled)

13. The personal care composition as claimed in claim 1, wherein the Moringa extract to the hydroxy fatty acid is present in a weight ratio ranging from 15:1 to 60:1.

14. The personal care composition as claimed in claim 1, wherein the Moringa extract is extracted from seeds of Moringa selected from Moringa oleifera Lam., Moringa pterygosperma, Moringa peregrina, Moringa concanensis and Moringa drouhardii.

15. The personal care composition as claimed in claim 1, wherein the Moringa extract is extracted from seeds of Moringa selected from Moringa oleifera Lam. and Moringa pterygosperma and wherein the Moringa extract is extract by aqueous solution.

16. The personal care composition as claimed in claim 1, wherein the hydroxy fatty acid is 12-hydroxystearic acid.

17. The personal care composition as claimed in claim 1, wherein the composition comprises 10 to 90% water by weight of the composition.

18. The personal care composition as claimed in claim 1, wherein the composition comprises 35 to 85% water by weight of the composition.

19. The personal care composition as claimed in claim 1, wherein the amount of the hydroxy fatty acid is in the range of 0.0001 to 0.01 by weight of the composition.

20. The personal care composition as claimed in claim 1, wherein the composition further comprises 0.1 to 10% of an organic sunscreen by weight of the composition.

21. The personal care composition as claimed in claim 19, wherein the organic sunscreen comprises of 2-ethylhexyl-p-methoxycinnamate, butylmethoxydibenzoylmethane, or a mixture thereof.
